(19) United States
(12) Patent Application Publication
(10) Pub. No.: US 2005/0149052 A1
(43) Pub. Date: Jul. 7, 2005

(54) Offset orthopaedic driver and method

(76) Inventor: Christopher M. Meek, Leesburg, IN (US)

Correspondence Address: ZIMMER TECHNOLOGY - REEVES, P. O. BOX 1268, ALEDO, TX, 76008, US

(21) Appl. No.: 10/750,439

(22) Filed: Dec. 31, 2003

(51) Int. Cl. A61B 17/58

(52) U.S. Cl. 606/104

(57) ABSTRACT

A driver and method are presented for driving fasteners inside of an orthopaedic implant.

### FIELD OF THE INVENTION

The invention relates to drivers for driving fasteners in an orthopaedic surgical environment. More particularly, the invention relates to a driver for driving fasteners inside of an orthopaedic implant.

### BACKGROUND

Degenerative and traumatic damage to the articular cartilage of the knee joint can result in pain and restricted motion. Prosthetic joint replacement is frequently utilized to alleviate the pain and restore joint function. In this procedure, the damaged compartments of the joint are cut away and replaced with prosthetic components. Typically a “C”-shaped femoral component is mated with the cut end of the femur and a tibial component is mated with the cut end of the tibia. In some situations, for example with osteoporotic bone, revision surgery, or where tumors must be removed, the bone may have to be resected more in one area than in another thus leaving the bone surface uneven. In these cases, the implants may be provided with modular augmentation blocks to build up the implant in areas where more bone has been removed. In other situations, convertible implants may be provided, such as femoral implants that may be converted from a less constraining configuration to a more constraining configuration by adding a modular intercondylar box component. In both of these situations, the modular component may be attached with fasteners, such as screws or nuts, that require a driver to engage the fasteners.

### SUMMARY

The present invention provides a driver and method for driving fasteners inside of an orthopaedic implant.

In one aspect of the invention, a driver for driving fasteners inside an orthopaedic implant includes an offset shaft. The shaft has a fastener engaging end, a driven end, and a longitudinal axis therebetween. The shaft has an intermediate offset portion that bends radially away from the axis. The engaging end and the driven end are coaxial for transmission of longitudinal forces along the axis and rotational forces about the axis from the driven end to the engaging end.

In another aspect of the invention, a driver is provided for driving fasteners inside an orthopaedic implant having a fastener receiving portion for receiving a fastener along a fastener axis and a portion of the implant overhanging the fastener receiving portion. The driver includes an offset shaft. The shaft has a fastener engaging end and a driven end. The fastener engaging end includes a longitudinal engaging end axis. The shaft has an intermediate portion between the fastener engaging end and the driven end that is offset radially from the engaging end axis such that the driver is engageable with a fastener engaged with the fastener receiving portion.

In another aspect of the invention, a driver is provided for driving fasteners inside the box area of a femoral prosthesis of an articulating knee joint implant. The implant has a patellar flange and a fastener receiving portion opposite the patellar flange for receiving a fastener along a fastener axis transverse to the patellar flange. The patellar flange has a patellar flange height measured from the fastener axis to an apex of the patellar flange. The driver includes a shaft having a fastener engaging end and a driven end. The engaging end has a longitudinal engaging end axis. The shaft has an intermediate portion between the engaging end and the driven end that is offset radially from the engaging end axis a distance equal to or greater than the patellar flange height such that the driver is engageable with a fastener along the fastener axis and the intermediate portion of the shaft clears the apex of the patellar flange when the driver is rotated.

In another aspect of the invention, a driver is provided for driving fasteners inside the box area of a femoral prosthesis of an articulating knee joint implant. The implant includes an anterior patellar flange, a distal condyle, and a posterior condyle forming an exterior articular surface. The implant also has an interior box area having an anterior box surface opposite the anterior patellar flange, a distal box surface opposite the distal condyle, and a posterior box surface opposite the posterior condyle. The distal box surface has a distal fastener receiving portion for receiving a fastener along a distal fastener axis and the posterior box surface has a posterior fastener receiving portion for receiving a fastener along a posterior fastener axis transverse to the anterior box surface. The patellar flange has a patellar flange height measured from the posterior fastener axis to an apex of the patellar flange perpendicular to the posterior fastener axis. The posterior condyle has a posterior condyle height measured from the distal box surface to an apex of the posterior condyle perpendicular to the distal box surface. The driver includes a shaft having a fastener engaging end, a driven end, and a shaft axis therebetween. The shaft has an intermediate portion between the fastener engaging end and the driven end. The intermediate portion has a first bend axially spaced a first distance from the fastener engaging end, a second bend offset radially from the shaft axis a second distance, and a third bend axially spaced a third distance from the fastener engaging end.

In another aspect of the invention, a combination includes an implant and a driver in which the driver includes an offset shaft configured to drive a fastener inside of the implant.

In another aspect of the invention, a method is provide for attaching an augmentation block to a femoral knee prosthesis.

### BRIEF DESCRIPTION OF THE DRAWINGS

Various embodiments of the present invention will be discussed with reference to the appended drawings. These drawings depict only illustrative embodiments of the invention and are not to be considered limiting of its scope.

FIG. 1 is a side plan view of a driver according to the present invention engaging a fastener to retain an augmentation block on a posterior box surface of a femoral knee implant;

FIG. 2 is a side plan view of the driver of FIG. 1 showing the driver rotated ninety degrees relative to its position in FIG. 1;

FIG. 3 is a front plan view of the driver of FIG. 1 oriented as in FIG. 2;

FIG. 4 is a side plan view of the driver of FIG. 1 showing the driver rotated ninety degrees relative to its position in FIG. 2; and

FIG. 5 is a side plan view of the driver of FIG. 1 showing the driver engaging a fastener to retain an augmentation block on a distal box surface of a femoral knee implant.

### DESCRIPTION OF THE ILLUSTRATED EMBODIMENTS

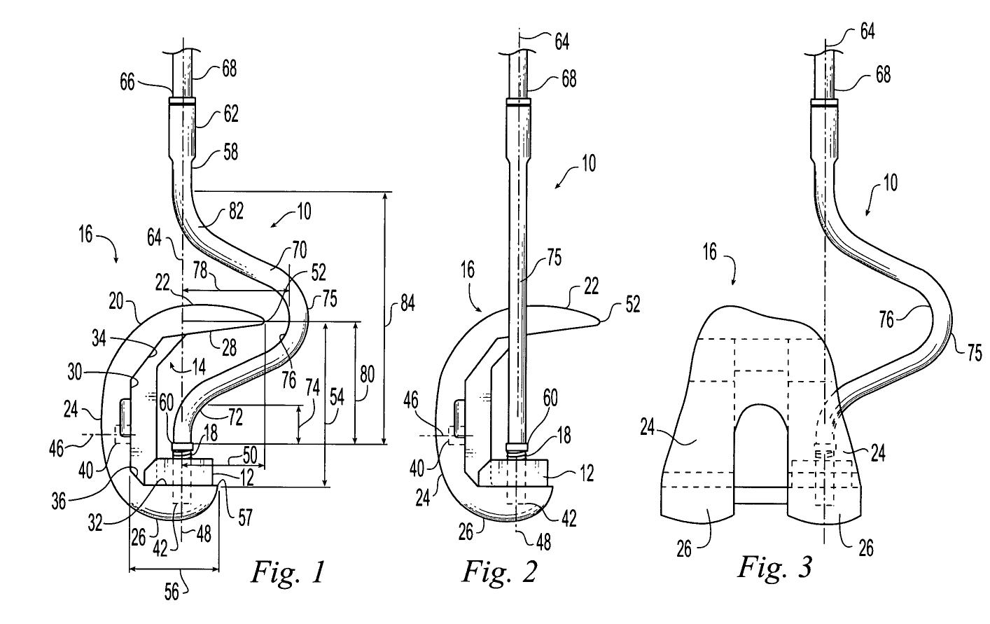

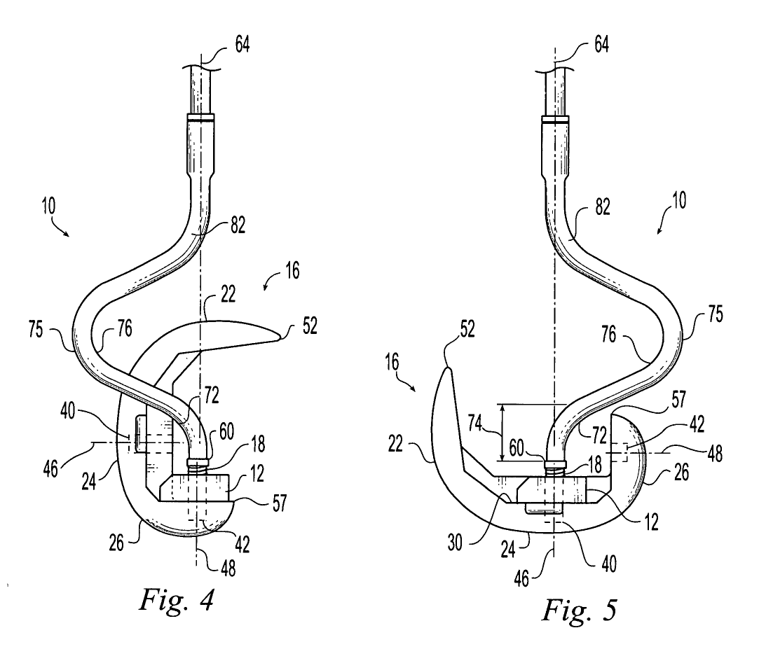

The driver of the present invention may be used with a variety of orthopaedic devices. In the illustrative examples of FIGS. 1-5, a driver 10 is shown in use to attach a modular augmentation block 12 to the interior box area 14 of a femoral knee component 16 using a fastener, such as a screw 18, driven through the block 12 and into the femoral component 16. It is contemplated that the driver 10 may also be configured to drive a nut onto a threaded post. Likewise, the driver 10 may be used to drive other types of fasteners and may be used with other kinds of orthopaedic devices.

Referring to FIG. 1, the exemplary femoral knee component 16 with which the driver 10 is used is generally a “C”-shaped member having an exterior articular surface 20. The articular surface 20 includes a patellar flange 22, a pair of distal condyles 24, and a pair of posterior condyles 26 for articulation with the tibia and patella. The interior box area 14 includes a faceted surface for seating on a cut end of a femur having corresponding faceted bone surfaces. The box area 14, includes an anterior surface 28 opposite the patellar flange 22, a distal surface 30 opposite the distal condyles 24, and a posterior surface 32 opposite the posterior condyles 26. An anterior chamfer surface 34 extends between the anterior and distal surfaces 28, 30, and a posterior chamfer surface 36 extends between the distal and posterior surfaces 30, 32. Distal and posterior threaded bores 40, 42 may be formed in the distal and posterior box surfaces 30, 32 to receive the screw 18. Each of the threaded bores 40, 42 has a fastener axis 46, 48 along the centerline of the bore 40, 42 and along which the screw 18 may be inserted.

The patellar flange 22 has a height 50 equal to the perpendicular distance from the posterior bore fastener axis 48 to the apex 52 of the patellar flange 22. The box 14 has an anteroposterior depth 54 equal to the distance from the posterior box surface 32 to the apex 52 of the anterior flange 22 along the fastener axis 48. The posterior condyle 26 has a height 56 equal to the distance from the distal box surface 30 to the apex 57 of the posterior condyle 26 measured perpendicular to the distal box surface 30. The augmentation block 12 may be attached to the box surfaces 30, 32 to thicken the femoral component 16 as necessary, such as to accommodate a bone defect. For example, the augmentation block 12 may be attached to the posterior box surface 32 by inserting the screw 18 through the augmentation block 12 and threading it into the threaded bore 42 along the fastener axis 48.

The driver 10 includes a shaft 58 having a fastener engaging end 60, a driven end 62, and a driver axis 64 extending between the fastener engaging end 60 and the driven end 62. The driver 10 may be driven by imparting driving forces to the driven end 62 directed along the driver axis 64 to translate the screw 18 along the fastener axis 48 and/or rotationally about the driver axis 64 to rotate the screw 18 about the fastener axis 48. In the exemplary embodiment, the fastener engaging end 60 and the driven end 62 are coaxial so that axial forces on the driven end 62 are aligned with the fastener axis 48 and press the fastener engaging end 60 of the driver 10 axially into engagement with the screw 18 without imparting a bending moment that would tend to disengage the driver 10 from the screw 18. For example, to drive the exemplary screw 18, axially forces press the screw 18 into engagement with the femoral component 16 and rotational forces turn the screw 18 into the threaded bore 42. The driven end 62 may have a handle or it may be connected to a rotary handpiece, torque wrench, ratchet wrench, or other instrument. For example, the shaft 58 may include a female socket 66 for receiving a drive shaft 68 from a ratchet wrench.

An intermediate portion 70 of the shaft 58 between the fastener engaging end 60 and the driven end 62 is offset radially from the driver axis 64 to provide clearance for inserting the fastener engaging end 60 into the box area 14 to engage the screw 18. In the exemplary embodiment, the intermediate portion 70 is offset a distance equal to or greater than the patellar flange 22 height 50 such that the driver 10 is engageable with a fastener along the fastener axis 48 and the intermediate portion 70 clears the apex 52 of the patellar flange 22.

The intermediate portion 70 of the shaft may be defined by three or more curves. In the illustrative example, the shaft 58 includes three curves. The first curve 72 is directed radially outwardly and begins the offset. The first curve 72 is preferably less than or equal to ninety degrees. The first curve 72 is spaced axially from the fastener engaging end 60 of the shaft 58 a first curve axial distance 74. The second curve 75 generally forms a “U”-shape with an inside apex 76 facing the driver axis 64 that is spaced radially from the driver axis 64 a second curve radial distance 78. The second curve 75 is preferably between ninety and one hundred eighty degrees and defines the maximum extent of the offset of the intermediate portion 70. The second curve 75 is spaced axially from the fastener engaging end 60 a second curve axial distance 80. The third curve 82 bends back into alignment with the driver axis 64 and is spaced axially from the fastener engaging end 60 a third curve axial distance 84.

As shown in FIGS. 1-4, the offset intermediate portion 70 permits the fastener engaging end 60 of the driver 10 to be inserted into the box area 14 and engaged with the screw 18 as it is threaded into the bore 42 in the posterior box surface 32. The second curve 75 of the intermediate portion 70 has a second curve radial distance 78 that is equal to or greater than the patellar flange 22 height 50 such that the driver 10 does not interfere with the apex 52 of the patellar flange 22. FIGS. 1-4 illustrate the driver 10 rotating the screw 18 while the offset portion 70 moves around the apex 52 of the patellar flange 22 without contacting the apex 52.

FIG. 5 shows the driver 10 being used to connect the augmentation block 12 to the distal box surface 30 by driving the screw 18 through the augmentation block and into the distal threaded bore 40. The first curve 72 axial spacing 74 is equal to or greater than the height 56 of the posterior condyles 26 so that the driver 10 does not interfere with the apex 57 of the posterior condyles 26.

The driver 10 of the illustrative examples includes an intermediate portion 70 defined by curves 72, 75, 82 that permit the driver 10 to engage a fastener inside of an implant such as inside the box area 14 of a femoral knee implant 16. The fastener engaging end 60 and driven end 62 of the driver 10 are preferably coaxial such that axial forces may be transmitted from the driven end 62 to the fastener without imparting bending moments. The intermediate portion 70 permits rotation of the driver 10 while avoiding impingement of the driver 10 on portions of the implant such as on the apex of the patellar flange 22 and the apex of the posterior condyles 26 of a femoral knee implant 16. In the exemplary embodiment, the curves 72, 75, 82 defining the intermediate portion 70 are positioned axially and radially such that the driver 10 specifically fits a particular femoral knee implant 16 geometry. It will be understood by those skilled in the art that the foregoing has described illustrative embodiments of the present invention and that variations may be made to these embodiments without departing from the spirit and scope of the invention defined by the appended claims.

1. A driver for driving fasteners inside an orthopaedic implant, the driver comprising:
a shaft having a fastener engaging end, a driven end, and a longitudinal axis therebetween, the shaft having an intermediate portion offset radially away from the axis, the engaging end and the driven end being coaxial for transmission of longitudinal forces along the axis and rotational forces about the axis from the driven end to the engaging end.

2. A driver for driving fasteners inside an orthopaedic implant having a fastener receiving portion for receiving a fastener along a fastener axis and a portion of the implant overhanging the fastener receiving portion, the driver comprising:
a shaft having a fastener engaging end and a driven end, the fastener engaging end having a longitudinal engaging end axis, the shaft having an intermediate portion between the fastener engaging end and the driven end that is offset radially from the engaging end axis such that the driver is engageable with a fastener engaged with the fastener receiving portion.

3. The driver of claim 2 wherein the driven end has a longitudinal driven end axis coaxial with the engaging end axis such that axial forces applied to the driven end are aligned with the engaging end.

4. The driver of claim 3 wherein the shaft is able to transmit torsional input from the driven end to the engaging end and the offset portion is shaped to extend around the overhanging portion such that the driver is rotatable to turn the fastener and the offset portion clears the overhanging portion of the implant.

5. A driver for driving fasteners inside the box area of a femoral prosthesis of an articulating knee joint implant, the implant having a patellar flange and a fastener receiving portion opposite the patellar flange for receiving a fastener along a fastener axis transverse to the patellar flange, the patellar flange having a patellar flange height measured from the fastener axis to an apex of the patellar flange, the driver comprising:
a shaft having a fastener engaging end and a driven end, the engaging end having a longitudinal engaging end axis, the shaft having an intermediate portion between the engaging end and the driven end that is offset radially from the engaging end axis a distance equal to or greater than the patellar flange height such that the driver is engageable with a fastener along the fastener axis and the intermediate portion of the shaft clears the apex of the patellar flange when the driver is rotated.

6. The driver of claim 5 wherein the driven end has a longitudinal driven end axis coaxial with the engaging end axis such that axial forces applied to the driven end are aligned with the engaging end.

7. A driver for driving fasteners inside the box area of a femoral prosthesis of an articulating knee joint implant, the implant having an anterior patellar flange, a distal condyle, and a posterior condyle forming an exterior articular surface, the implant having an interior box area having an anterior box surface opposite the anterior patellar flange, a distal box surface opposite the distal condyle, and a posterior box surface opposite the posterior condyle, the distal box surface having a distal fastener receiving portion for receiving a fastener along a distal fastener axis and the posterior box surface having a posterior fastener receiving portion for receiving a fastener along a posterior fastener axis transverse to the anterior box surface, the patellar flange having a patellar flange height measured from the posterior fastener axis to an apex of the patellar flange perpendicular to the posterior fastener axis, the posterior condyle having a posterior condyle height measured from the distal box surface to an apex of the posterior condyle perpendicular to the distal box surface, the driver comprising:
a shaft having a fastener engaging end, a driven end, and a shaft axis therebetween, the shaft having an intermediate portion between the fastener engaging end and the driven end, the intermediate portion having a first bend axially spaced a first distance from the fastener engaging end, a second bend offset radially from the shaft axis a second distance, and a third bend axially spaced a third distance from the fastener engaging end.

8. The driver of claim 7 wherein the second distance is equal to or greater than the patellar flange height such that the driver is engageable with a fastener along the posterior fastener axis with the intermediate portion of the shaft clearing the apex of the patellar flange.

9. The driver of claim 7 wherein the first distance is equal to or greater than the posterior condyle height such that the driver is engageable with a fastener along the distal fastener axis with the intermediate portion of the shaft clearing the apex of the posterior condyle.

10. A combination of an implant and a driver, the combination comprising:
a femoral prosthesis of an articulating knee joint implant, the implant having a patellar flange and a fastener receiving portion opposite the patellar flange for receiving a fastener along a fastener axis transverse to the patellar flange, the patellar flange having a patellar flange height measured from the fastener axis to an apex of the patellar flange; and
a driver including a shaft having a fastener engaging end and a driven end, the engaging end having a longitudinal engaging end axis, the shaft having an intermediate portion between the engaging end and the driven end that is offset radially from the engaging end axis a distance equal to or greater than the patellar flange height such that the driver is engageable with a fastener along the fastener axis and the intermediate portion of the shaft clears the apex of the patellar flange when the driver is rotated.

11. A method of attaching an augmentation block to a femoral knee prosthesis, the method comprising:
providing a femoral knee prosthesis having a patellar flange and a fastener receiving portion opposite the patellar flange for receiving a fastener along a fastener axis transverse to the patellar flange, the patellar flange having a patellar flange height measured from the fastener axis to an apex of the patellar flange;
providing an augmentation block positionable adjacent the fastener receiving portion;
providing a fastener engageable with the fastener receiving portion to fasten the augmentation block in place;
providing a driver having a shaft with a fastener engaging end and a driven end, the engaging end having a longitudinal engaging end axis, the shaft having an intermediate portion between the engaging end and the driven end that is offset radially from the engaging end axis a distance equal to or greater than the patellar flange height;
positioning the augmentation block adjacent the fastener receiving portion;
engaging the fastener with the fastener receiving portion;
engaging the driver with the fastener along the fastener axis; and
rotating the driver shaft to drive the fastener while clearing the apex of the patellar flange with the intermediate portion of the shaft.
